(19) United States
(12) Patent Application Publication
(10) Pub. No.: US 2025/0228762 A1
(43) Pub. Date: Jul. 17, 2025

(54) COSMETIC COMPOSITION COMPRISING LUTEIN AND A POLYSACCHARIDE

(71) Applicant: ALGAKTIV, S.L., Barcelona (ES)

(72) Inventors: Noemí GARCÍA-DELGADO BANCHS, Esplugues del Llobregat, Barcelona (ES); Jaume MERCADÉ ROCA, Barcelona (ES); Eugènia RUIZ CÁNOVAS, Granollers, Barcelona (ES); Joan TÀRRAGA ROSELL, Beders, Lleida (ES); Jordi AYATS FORRELLAT, Terrassa, Barcelona (ES)

(21) Appl. No.: 18/853,918

(22) PCT Filed: Apr. 3, 2023
(86) PCT No.: PCT/EP2023/058620

§ 371 Date: Oct. 3, 2024

(30) Foreign Application Priority Data
Apr. 4, 2022 (EP) 22382315.4

(51) Int. Cl. A61K 8/9722; A61K 8/14; A61Q 19/08

(52) U.S. Cl. CPC A61K 8/9722; A61K 8/14; A61Q 19/08; A61K 2800/75; A61K 2800/805

(57) ABSTRACT

The invention relates to algal-derived compositions comprising lutein and polysaccharides. The invention also relates to the use of the compositions as foodstuff, cosmeceutical, cosmetic, nutraceutical, nutritional supplement or pharmaceutical composition.

### FIELD OF THE INVENTION

The invention relates to the field of natural products and uses thereof in cosmetic methods as well as in the preparation of foodstuff, cosmeceutical, nutritional supplement, nutraceutical or pharmaceutical composition.

### BACKGROUND OF THE INVENTION

Topical administration of retinoids is commonly used for the treatment of different ailments as well as for cosmetic purposes, e.g. as anti-aging compounds. However, many patients undergoing topical retinoid therapy experience erythema, scaling, dryness, burning and pruritus. These symptoms characterize the retinoid-specific irritant contact dermatitis commonly termed ‘retinoid dermatitis. The clinical picture of retinoid dermatitis varies in intensity, and many patients who would benefit from this type of therapy discontinue treatment due to discomfort. Erythema, scaling/peeling, dryness, burning and pruritus characterize the irritation.

These effects seem to be related to the activation of the retinoic acid receptors by the carotenoids, of which there exist three retinoic acid receptors (RARs) α, β, and γ which are members of the nuclear receptor superfamily. It has been established that cutaneous irritation is not related to RARα agonist activity. Since RARγ is more expressed than RAR β a well-established correlation of irritation with RARγ has been established both in in vitro and in vivo models

There is a need in the art of cosmetic compositions having similar effect to retinol on skin but without producing irritating effects.

### SUMMARY OF THE INVENTION

In a first aspect, the invention relates to a combination comprising lutein and a polysaccharide, wherein the combination is characterized in that it has
(i) a content of apocarotenoids which is of at least 0.00005% % (w/w) and/or
(ii) a content of chlorophyll which is below 0.05%

In another aspect, the invention relates to a method for preparing a combination according to the invention comprising the steps of:
a. Preparing a first extract containing lutein and apocarotenoids from a first algal biomass preparation by a process comprising the steps of:
   (i) Extracting the first algal biomass with an organic solvent,
   (ii) Applying saponification conditions to the extract obtained in step (i),
   (iii) Removing negatively charged compounds resulting from the saponification carried out in step (ii) and
   (iv) Treating the fraction obtained in step (iii) under oxidative conditions in order to convert part of the carotenoids found in the fraction to apocarotenoids,
b. Preparing a second extract containing polysaccharides from a second algal biomass preparation by a process comprising the steps of:
   (i) disrupting the cell wall of the second algal biomass,
   (ii) recovering the alkali-insoluble fraction from the fraction obtained in step (i) and
   (iii) solubilizing the alkali-insoluble fraction in acidic media and
c. Combining the fractions obtained in steps a. and b.

In further aspects, the invention relates to a foodstuff, cosmeceutical, cosmetic, nutraceutical, nutritional supplement or pharmaceutical composition comprising the combination according to the invention.

### DESCRIPTION OF THE FIGURES

FIG. 1: UV spectra of some selected lutein derivatives detected by HPLC.

FIG. 2: shows the dose-response curve of lutein action on RAR alpha. ATRA was the control agonist of the assay (EC50 2.37 nM). Error bars indicate standard deviation.

FIG. 3: shows the dose-response curve of lutein action on RAR beta. ATRA was the control agonist of the assay (EC50 0.46 nM). Error bars indicate standard deviation.

FIG. 4: shows the dose-response curve of lutein action on RAR gamma. ATRA was the control agonist of the assay (EC50 0.131 nM). Error bars indicate standard deviation.

FIG. 5: The graph reports the mean data obtained at each experimental check for the monitored parameter. Data are expressed as mean±SEM. Above the error bar the intra-group (black asterisks) and inter-group (red asterisks) statistical analysis is reported as follow: *p<0.05; **p<0.01; ***p<0.001

FIG. 6: The graph reports the mean data obtained at each experimental check for the monitored parameter. Data are expressed as mean±SEM. Above the error bar the intra-group (black asterisks) and inter-group (red asterisks) statistical analysis is reported as follow: *p<0.05; **p<0.01; ***p<0.001

FIG. 7: The graphs report the percentage of subjects related to the effect. The positive effect of the product on the evaluated parameter is confirmed if more than 50% of the subjects register an improvement

### DESCRIPTION OF THE INVENTION

The authors of the present invention have found that lutein is a RAR agonist which acts specifically via the RAR-beta receptors, whereas the activity towards the alpha and gamma receptors is negligible. Moreover, the authors have also found that compositions derived from algae which contain lutein and the algal polysaccharides are formulations that can be used when the cosmetic or therapeutic effects of lutein are desired. These compositions

Accordingly, in a first aspect, the invention relates to a combination comprising lutein and a polysaccharide, wherein the combination is characterized in that it has
(i) a content of apocarotenoids which is of at least 0.00005% % (w/w) and/or
(ii) a content of chlorophyll which is below 0.05%

As used herein, the term combination is used to define any composition-of-matter, which contains, either jointly or separately, lutein and a polysaccharide. The term combination includes a combined mixture composed of separate formulations of the components (i) and (ii), such as a “tank-mix”, and a composition in which all the components form part of the same mixture. It will be understood that when the combination is provided as a mixture of components (i) and (ii), then the other components (apocarotenoids and chlorophyll if present) will be forming part of the same mixture. However, in those cases wherein the components (i) and (ii) are provided in separate containers, then the remaining components (apocarotenoids and chlorophyll if present) may be forming part of the container containing component (i) or of the container containing component (ii). In some embodiments, the lutein and the carotenoids are found in one container and the polysaccharides in the second container. The chlorophyll, if present, may be present in the first, in the second or in the third container. Whenever the combination is provided as a mixture of all components, it will be understood that, when in use, all the components will be administered simultaneously. However, if those embodiments wherein the different components of the combination are formulated in different containers, then the combination may be formulated for its simultaneous, separate or sequential administration. Whenever the components of the combination are provided as a mixture of at least components (i) and (ii), then the terms “combination” and “composition” can be used interchangeably.

The term “lutein”, as used herein, refers to the compound β,ε-Carotene-3,3′-diol or (1R,4R)-4-{(1E,3E,5E,7E,9E,11E,13E,15E,17E)-18-[(4R)-4-Hydroxy-2,6,6-trimethylcyclohex-1-en-1-yl]-3,7,12,16-tetramethyloctadeca-1,3,5,7,9,11,13,15,17-nonaen-1-yl}-3,5,5-trimethylcyclohex-2-en-1-ol, and defined under the CAS number 127-40-2.

In one embodiment, the combination according to the invention has a lutein content which is at least 0.00005% (w/w). In some embodiments, the lutein content in the combination is of between 0.00005% (w/w)-5% (w/w), between 0.05% (w/w)-5% (w/w) or of between 2% (w/w)-5% (w/w) with respect to the total weight of the composition.

The term “polysaccharide”, as used herein, refers to a long chain polymeric carbohydrate composed of monosaccharide units bound together by glycosidic linkages. Suitable polysaccharides that can be found in the combinations according to the present invention include, without limitation, starch, cellulose, galacturonic acid-rich polysaccharides and glucosamine-rich polysaccharides.

As used herein, the term “starch” is used to define a glucose polymer in which glucopyranose units are bonded by alpha-linkages and which is made up of a mixture of amylose (15-20%) and amylopectin (80-85%). Amylose consists of a linear chain of several hundred glucose molecules, and Amylopectin is a branched molecule made of several thousand glucose units (every chain of 24-30 glucose units is one unit of Amylopectin).

As used herein “cellulose” refers to a polymer composed of a linear chain of several hundred to many thousands of β(1→4) linked D-glucose units

The term “galacturonic acid-rich polysaccharide”, as used herein, refers to any polysaccharide in which a substantial percentage of the integrating monomers are galacturonic acid. Galacturonic acid is an oxidized form of D-galactose having a CAS number 685-73-4. In a preferred embodiment, the galacturonic acid-rich polysaccharide is pectin, which includes:
Homogalacturonans which consists of linear chains of α-(1-4)-linked D-galacturonic acid.
Substituted galacturonans which consists of chains of α-(1-4)-linked D-galacturonic containing saccharide appendant residues (such as D-xylose or D-apiose in the respective cases of xylogalacturonan and apiogalacturonan) branching from a backbone of D-galacturonic acid residues.
Rhamnogalacturonan I pectins (RG-I), which contain a backbone of the repeating disaccharide: 4)-α-D-galacturonic acid-(1,2)-α-L-rhamnose-(1. And containing sidechains of various neutral sugars braching from many of the rhamnose residues, such as D-galactose, L-arabinose and D-xylose
Rhamnogalacturonan II (RG-II), which is a highly branched polysaccharide consisting of a backbone made exclusively of D-galacturonic acid units.

The term a “glucosamine-rich polysaccharide” refers to any polysaccharide in which a substantial percentage of the integrating monomers are D-glucosamine. D-glucosamine refers to a glucose molecule in which position 3 is an amine group.

In some embodiments, the glucosamine-rich polysaccharide is selected from the group consisting of chitin, chitosan or a combination thereof.

The term “chitin”, as used herein, relates to a β(1-4) polymer of N-acetyl-D-glucosamine that is the major structural component of the exoskeleton of invertebrates, cuticles of insects and the cell walls of fungi. Chitin is a linear, highly crystalline homo polymer of β-1,4 N-acetyl glucosamine (GlcNAc), that consists of β-1,4-linked N-acetyl glucosamine residues that are arranged in antiparallel (α), parallel (β) or mixed (γ, two parallel strands alternate with a single anti-parallel strand) strands, with the (α) configuration being the most abundant. In most organisms, chitin is cross-linked to other structural components, such as proteins and glucans. Chitin is represented by the following formula:

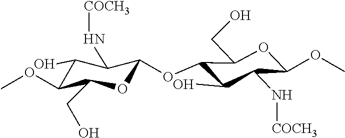

In a particular embodiment, the degree of polymerization of the chitin according to the invention ranges from 50 to 500, preferably between 100 and 250. In a particular embodiment, the chitin according to the invention shows a polydispersity index less than or equal to 2.0, preferably ranging between 1.0 and 2.0.

The term “degree of polymerization”, as used herein, relates to the number of monomeric units in a macromolecule or polymer. Methods to determine the degree of polymerization are known by the skilled person and are based, mainly, in number average degree of polymerization and weight average degree of polymerization. Number average degree of polymerization is found by finding the weighted mean of mole fraction; weight average degree of polymerization is found by finding the weighted mean of weight fraction.

The term “polydispersity index”, also known as “dispersity”, relates to a measure of the width of molecular weight distributions. This parameter measures the heterogeneity of sizes of molecules or particles in a mixture. Methods to determine dispersity are known by the skilled person and include, without limitation, size exclusion chromatography, light scattering measurement and mass spectrometry (MALDI, electrospray ionization).

The term also refers to chitin derivatives. Chitin derivatives according to the present invention include, without limitation, chitin phosphate, chitin phosphate sulphate, chitin ethylene glycol, aminoethyl-chitin, carboxymethyl chitin, chitosan hydrogel, and hydroxyethyl chitin. Chitin derivatives can be obtained from chitin by methods known by the skilled person.

In some embodiments, the polysaccharide is chitin which is characterized by a degree of polymerization of 50-500 and/or a polydispersity index of less than or equal to 2.0.

The term “chitosan”, as used herein, relates to a derivative of chitin obtained by deacetylation of chitin in the solid state under alkaline conditions (such as concentrated NaOH) or by enzymatic hydrolysis in the presence of a chitin deacetylase. It is a linear polysaccharide composed of randomly distributed β-(1-4)-linked D-glucosamine (deacetylated unit) and N-acetyl-D-glucosamine (acetylated unit), characterized by its average molecular weight and its degree of acetylation (proportion of acetylated glucosamine units along the polymer backbone). Chitosan is represented by the following general formula:

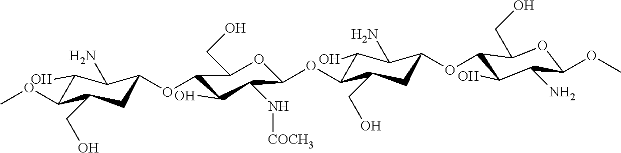

In a particular embodiment, the molecular weight of chitosan according to the invention is between 10 and 60 kDa, more preferably between 15 and 50 kDa. In a particular embodiment, the degree of acetylation ranges from 1 to 40%, preferably between 7 and 35%. In a particular embodiment, the degree of polymerization of chitosan according to the invention ranges from 50 to 500, preferably between 100 and 250. In a particular embodiment, the chitosan according to the invention shows a polydispersity index less than or equal to 2.0, preferably ranging between 1.0 and 2.0.

The term “molecular weight”, as used herein, relates to the average molar mass of a molecule. Unlike small molecules, the molecular weight of a polymer is not one unique value. Rather, a given polymer will have a distribution of molecular weights depending for example on the way the polymer is produced. Therefore, as it is used herein, the term molecular weight for polymers refers to the distribution of molecular weight, or of the average molecular weight. Methods to determine the molecular weight are known by the skilled person and include, without limitation, 1H-NMR.

The term “degree of acetylation”, as used herein, relates to presence of acetyl functional groups in a compound. Removal of said acetyl functional groups is known as deacetylation. Methods to determine the degree of acetylation/deacetylation are known by the skilled person and include, without limitation, nuclear magnetic resonance (NMR) spectroscopy.

In some embodiments, the polysaccharide is chitosan which is characterized by a molecular weight of 10-60 kDa, a degree of acetylation of 1-40%, a degree of polymerization of 50-500 and/or a polydispersity index of less than or equal to 2.0.

Particularly preferred methods according to the invention for determining the degree of acetylation, the average molecular weight and polydispersity for chitosan, as well as for chitin, are known in the art, for example WO2016096986A1.

The term “chitosan” also includes chitosan derivatives such as PEG-chitosan (copolymer), chitosan azide, N-phthaloyl chitosan, chitosan-C(6)-MPEG (copolymer), chitosan adipate, chitosan fumarate, chitosan lactate, chitosan acetate, chitosan hydrochloride, carboxymethylchitosan, N-sulfonato-N,O-carboxymethylchitosan, chitosan ascorbate, chitosan malate, chitosan glutamate, trimethyl chitosan (TMC), aryl chitosan, thiolated chitosan, N-succinyl-chitosan (Suc-Chi), thiosemicarbazone chitosans, N,O-carboxymethylchitosan(NOCC) and hydroxyl propylatedchitosan(HPC), N-trimethylene chloride chitosan), chitosan phthalate, and trimethyl ammonium chitosan. Chitosan derivatives can be obtained from chitosan by methods known by the skilled person.

In some embodiments, the content of polysaccharides in the combination is at least 90% (w/w), at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.7%, at least 99.8% or at least 99.9%, all values in w/w with respect to the total weight of the combination.

In a preferred embodiment, the combination of the invention comprises lutein and starch. In another preferred embodiment, the combination of the invention comprises lutein and pectin. In another preferred embodiment, the combination of the invention comprises lutein and cellulose. In another preferred embodiment, the combination of the invention comprises lutein and chitin. In another preferred embodiment, the combination of the invention comprises lutein and chitosan. In another preferred embodiment, the combination of the invention comprises lutein, chitin and chitosan.

In some embodiments, the weight ratio of lutein to polysaccharides by weight in the combinations according to the invention is of about 0.00001:1; 0.00005:1; 0.0001:1; 0.0005:1; 0.001:1; 0.05; 1; 0.1:1 or 1:1.

The combinations of the invention are characterized in that they contain at least 0.00005% (w(w) of apocarotenoids with respect to the total weight of the combination.

The term “apocarotenoid”, as used herein, refers to a cleavage product derived from one or more carotenoids. Examples of apocarotenoids include, but are not limited to, abscisic acid, apocarotenal, bixin, beta-ionone, beta-cyclocitral, crocetin, safranal, dihydro-beta-Ionone, dimethyl-beta cyclocitral, dihydroactinidiolide (DHAD), ethyl ester of beta-apo-8′-carotenic acid, α-ionone, pseudoionone, peridinin, apo-10-carotenal (Apo10), apo-12-carotenal (Apo12), apo-14-carotenal (Apo14), apo-14-carotenoic acid, apo-14-carotenol, apo-16-carotenal (Retinal), retinol, retinoic acid (RA), dehydroretinal, tretinoin, isotretinoin, alitretinoin, etretinate, acitretin, adapalene, bexarotene, tazaotene, and/or any derivatives (including functional derivatives) and/or analogues and/or combinations thereof.

The content of apocarotenoids in the combination according to the invention can be defined either by its percent content with respect to the total weight of the combination or by its percent content or ratio with respect to the lutein present in the combination.

In one embodiment, the content of apocarotenoids in the combination of the invention is of between 0.00005%-5% (w/w), more preferably, between 0.00005-2% (w/w) and even more preferably between 0.0005-0.05% (w/w).

In some embodiments, the ratio of lutein to apocarotenoids by weight in the combination of the invention is of about 0.001:1; 0.005:1; 0.01:1, 0.05:1; 0.1:1, 0.5:1, 1:1, 1:0.5; 1:0.1; 1:0.5; 1:0.01; 1:0.05 or 1:0.001.

The relative content of apocarotenoids with respect to lutein can be determined as explained below by chromatographic separation of the fraction containing the lutein and the apocarotenoids, determining the areas of the peaks corresponding to lutein and to each of the apocarotenoids and determining the ratio of the aggregate area of all peaks corresponding to apocarotenoids to the area of the peak corresponding to lutein.

The term “chlorophyll”, as used herein, includes pigments comprising a porphyrin ring, found in cyanobacteria, algae and plants, and that are involved in the photosynthesis, including without limitation chlorophyll A, chlorophyll B, and chlorophyll C.

The combinations of the invention are characterized in that they contain a content in chlorophyll which is lower than 0.05% (w/w) with respect to the total weight of the combination. In some embodiments, the content in chlorophyll in the compositions according to the invention is of less than 0.045%, less than 0.04%, less than 0.035%, less than 0.030%, less than 0.025%, less than 0.002%, less than 0.015%, less than 0.01% or lower.

In some embodiments, the combinations of the invention are provided so that at least one of the components (i) or (ii) are encapsulated in liposomes.

As used herein, the term “liposome” is used to define a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. Liposomes provided herein include unilamellar liposomes, multilamellar liposomes and multivesicular liposomes. Liposomes provided herein may be positively charged, negatively charged or neutrally charged. In certain embodiments, the liposomes are neutral in charge.

Liposome used according to the present embodiments can be made by different methods, as would be known to one of ordinary skill in the art.

The size of a liposome varies depending on the method of synthesis. Liposomes in the present embodiments can be a variety of sizes. In certain embodiments, the liposomes are small, e.g., less than about 500 nm, less than about 400 nm, less than about 100 nm, about 90 nm, about 80 nm, about 70 nm, about 60 nm, or less than about 50 nm in external diameter. Such liposome formulations may also be defined by particle charge (zeta potential) and/or optical density (OD). For instance, a DOTAP: cholesterol liposome formulation will typically comprise an OD400 of less than 0.45 prior to nucleic acid incorporation. Likewise, the overall charge of such particles in solution can be defined by a zeta potential of about 50-80 mV.

In certain embodiments, the lipid-based nanoparticle is a neutral liposome (e.g., a DOPC liposome). “Neutral liposomes” or “non-charged liposomes”, as used herein, are defined as liposomes having one or more lipid components that yield an essentially-neutral, net charge (substantially non-charged). By “essentially neutral” or “essentially non-charged”, it is meant that few, if any, lipid components within a given population (e.g., a population of liposomes) include a charge that is not canceled by an opposite charge of another component (/.<., fewer than 10 percent of components include a non-canceled charge, more preferably fewer than 5 percent, and most preferably fewer than 1 percent). In certain embodiments, neutral liposomes may include mostly lipids and/or phospholipids that are themselves neutral under physiological conditions (/.<., at about pH 7).

Liposomes of the present invention may comprise a phospholipid. In certain embodiments, a single kind of phospholipid may be used in the creation of liposomes (e.g., a neutral phospholipid, such as DOPC, may be used to generate neutral liposomes). In other embodiments, more than one kind of phospholipid may be used to create liposomes.

Phospholipids include, for example, phosphatidylcholines, phosphatidylglycerols, and phosphatidylethanolamines; because phosphatidylethanolamines and phosphatidyl cholines are non-charged under physiological conditions (i.e., at about pH 7), these compounds may be particularly useful for generating neutral liposomes. In certain embodiments, the phospholipid DOPC is used to produce non-charged liposomes. In certain embodiments, a lipid that is not a phospholipid (e.g., a cholesterol) may be used

Phospholipids include glycerophospholipids and certain sphingolipids. Phospholipids include, but are not limited to, di oleoylphosphatidyly choline (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), I-palmitoyl-2-myristoyl phosphatidylcholine (“PMPC”), I-palmitoyl-2-stearoyl phosphatidylcholine (“PSPC”), I-stearoyl-2-palmitoyl phosphatidylcholine (“SPPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), distearoyl sphingomyelin (“DSSP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPP A”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), I,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine.

In some embodiments, the combination is a composition containing liposomes and polysaccharides wherein the liposomes encapsulate the lutein and the apocarotenoids but do not encapsulate the polysaccharides. In this case, the composition of the invention comprises liposomes encapsulating the lutein and the apocarotenoids and the polysaccharides are in soluble form.

In some embodiments, the combination is a composition comprising liposomes, wherein the liposomes encapsulate the lutein, the apocarotenoids, the polysaccharides and the chlorophyll if present.

In some embodiments, the combination is a composition comprising a first population of liposomes in which the lutein and apocarotenoids are encapsulated and a second population of liposomes in which the polysaccharides are encapsulated. If present the chlorophyll may be present in the first population of liposomes, in the second population of liposomes or in both the first and second populations. In any of these three types of compositions, the chlorophyll may be also be present in soluble form in the media in which the liposomes are suspended.

In some embodiments, the combination of the invention may be in the form of a water-soluble formulation or in the form of an emulsion.

### Method for Preparing the Combination of the Invention

In another aspect, the invention relates to a method for preparing a combination according to the invention comprising the steps of:
a. Preparing a first extract containing lutein and apocarotenoids from a first algal biomass preparation by a process comprising the steps of:
   (i) Extracting the first algal biomass with an organic solvent,
   (ii) Applying saponification conditions to the extract obtained in step (i),
   (iii) Removing negatively charged compounds resulting from the saponification carried out in step (ii) and
   (iv) Treating the fraction obtained in step (iii) under oxidative conditions in order to convert part of the carotenoids found in the fraction to apocarotenoids,
b. Preparing a second extract containing polysaccharides from a second algal biomass preparation by a process comprising the steps of:
   (i) disrupting the cell wall of the second algal biomass,
   (ii) recovering the alkali-insoluble fraction from the fraction obtained in step (i) and
   (iii) solubilizing the alkali-insoluble fraction in acidic media and
c. Combining the fractions obtained in steps a. and b.

In a first step, a first preparation of algal biomass is treated in order to obtain the first component of the combination of the invention, namely, the lutein and apocarotenoids.

The term “algae”, as used herein, relates to a large and diverse group of simple, typically autotrophic organisms, ranging from unicellular to multicellular forms, including both macroalgae and microalgae, i.e., microscopic algae, typically found in freshwater and marine systems. In the context of the present invention, the alga is a microalga, particularly a chitin and/or chitosan producing microalga.

The term “biomass”, as used herein, includes biological material comprising, or deriving from, living or recently living organisms. By extension, the term includes not only the biological material or organic matter which constitutes an organism, but also the biological material or organic matter generated in a biological process, spontaneous or not spontaneous (i.e., provoked).

As used herein, the term “algal biomass” refers to a population of microbial cells.

In a preferred embodiment, the algal biomass is from a species of the genus Chlorella. Preferably the microalga belonging to the Chlorella genus is selected from the group consisting of Chlorella saccharophila, Chlorella vulgaris (CS41), Chlorella sorokiniana or Chlorella sp.,

The first step is carried out in four different substeps, namely:
(i) Extracting the first algal biomass with an organic solvent,
(ii) Applying saponification conditions to the extract obtained in step (i),
(iii) Removing negatively charged compounds resulting from the saponification carried out in step (ii) and
(iv) Treating the fraction obtained in step (iii) under oxidative conditions in order to convert part of the carotenoids found in the fraction to apocarotenoids,

Step a(i) involves the extraction of the algal biomass with an organic solvent. In one embodiment, the step is carried out without a previous treatment of the biomass to disrupt the cell wall or to lyse the cells.

As used herein, organic solvent refers to an organic compound, different from an oil and different from a surfactant (e.g., an organic solvent is generally non-surface active and non-amphiphilic), which is miscible in the liquid extraction medium and together with surfactant and water of a liquid extraction medium may form a liquid extraction medium or a portion of a liquid extraction medium (e.g., homogenous liquid or liquid phase of an extraction medium) that dissolves the target algal material. The term “organic solvent” will be readily known to those skilled in the art, but may include chemical solvents such as acetone, acetonitrile, benzene, chloroform, 1,4-dioxane, diethyl ether, dichloromethane, dimethylacetamide, N,N-dimethylformamide, dimethyl sulfoxide, ethanol, ethyl acetate, hexanes, isopropanol, methanol, N-methylpyrolidone, pyridine, tetrahydrofuran, toluene. In one embodiment, the organic solvent used in the extraction step a(i) is ethanol.

Typical steps of a chemical extraction process may include a step of mechanically processing an amount of algal material, and a step (optionally in combination with the mechanical processing step) of chemically extracting algal material, normally along with an amount of endogenous non-target plant material, from the mechanically processed algal material or a derivative thereof. The mechanical processing and chemical extraction process may be performed by any useful mechanical processing and chemical extraction techniques, in a useful sequence. The steps can include contacting the algal material with a liquid extraction medium as described herein (or individual components of a liquid extraction medium), e.g., that contains: a non-aqueous phase that may contain one or more surfactants, dissolved water, and other dissolved exogenous processing ingredients or dissolved ingredients such as acid, base, hydrotrope, solubilizing agent, and (while not required or necessarily preferred) an amount of dissolved exogenous oil, dissolved exogenous organic solvent, or two or more of these. If a sufficiently high amount of water is combined with the non-aqueous phase, the liquid extraction medium can be in the form of a multiple-phase liquid that includes the non-aqueous and an aqueous phase that is made mostly of water, e.g., as multiple phases of an emulsion.

In some embodiments, an organic solvent can be added to a microbial cell composition, a lysed cell composition, or a demulsified cell composition. In such embodiments, the organic solvent is added in a concentration less than 5 percent, less than 4 percent, less than 3 percent, less than 2 percent, less than 1 percent, less than 0.5 percent, less than 0.1 percent, or less than 0.05 percent by volume.

Step a(ii) comprises applying saponification conditions to the extract obtained in step (i). The term “saponification” as used herein, refers to a process by which a fat, an oil, or lipid is turned into soap and alcohol by the action of aqueous alkali acting on an ester bond found in the fat, oil or lipid.

In some embodiments, the extract is contacted with a base for a period of time and then heated, agitated, or a combination thereof until saponification is achieved. In some embodiments, the base has a pKb of 1 to 12, 1 to 10, 1 to 8, 1 to 6, 1 to 5, 2 to 12, 2 to 10, 2 to 8, 2 to 6, 2 to 5, 3 to 10, 3 to 6, 3 to 5, 4 to 10, 4 to 8, 4 to 6, 5 to 10, or 5 to 8. As used herein, the term “pKt,” refers to the negative logarithm of the base association constant, Kb, of the base. Kb refers to the equilibrium constant for the ionization of the base in water.

Bases suitable for use with the present invention include, but are not limited to, hydroxide bases (e.g., LiOH, NaOH, KOH, Ca(OH)₂, and the like, and combinations thereof), carbonate bases (e.g., Na₂CO₃, K₂CO₃, MgCO3, and the like, and combinations thereof), bicarbonate bases (e.g., LiHCO3, NaHCO₃, KHCO₃, and the like, and combinations thereof), ammonia or quaternary ammonium salts.

The amount of base of use in steps (a)(ii) is sufficient to maintain a pH of at least about 7.0, e.g. at least about 7.5, 8.0, 8.5, 9.0, 9.5, 10.0, 10.5, 11.0, 11.5, 12.0, 12.5, 13.0, 13.5, 14.0, or 14.5 in the mixture for the duration of steps (a)(ii). In certain embodiments, the reaction is allowed to proceed until substantially, e.g. essentially, all of the chlorophyll in the mixture is converted to a magnesium chlorophyllin alkali metal salt or alkali earth metal salt.

In some embodiments, a base is added in an amount of about 2 percent to about 10 percent, about 2 percent to about 9 percent, about 2 percent to about 8 percent, about 2 percent to about 7 percent, about 2 percent to about 6 percent, about 3 percent to about 6 percent, about 4 percent to about 6 percent, about 5 percent to about 6 percent, about 2 percent to about 5 percent, about 2 percent to about 4 percent, about 2 percent to about 3 percent, about 3 percent to about 5 percent, about 3 percent to about 4 percent, or about 4 percent to about 5 percent by weight (or volume) of the cell broth to raise the pH.

In the method according to the present invention, the saponification applied in step (ii) to the extract obtained in step (i) results in the conversion of the triglycerides derived from the membranes of the algal biomass into the corresponding alcohols and fatty acid salts. Saponification also affects chlorophyll. Chlorophyl is formed by a chlorin ring attached to phythyl chain by an ester group. When the chlorophyll is placed under saponification conditions, the ester bond connecting the chlorin ring and the phythyl group is cleaved, thereby releasing the negatively-charged chlorophyll moiety. The negatively charged fatty acids and the negatively charged chlorin ring can then be removed based on its net electrical charge.

Expressed numerically, the reaction is allowed to proceed until at least about 80 percent, e.g. at least about 85 percent, 90 percent, 95 percent, 99 percent or 100 percent completion.

The progress of this reaction may be monitored by routine means, e.g. monitoring absorbance of the reaction mixture at 405 nm and/or 653 nm, e.g. at a pH adjusted to 7.5 to 9.0. It will be seen therefore that, in certain embodiments, the amount of base and any additional salt used should be in excess of the amount of chlorophyll which is present in the extract.

In one embodiment, the saponification is carried out by adding an immobilized strong base to the extract. Immobilization is usually achieved by coupling the strong base to a solid support.

Solid supports suitable for the methods disclosed herein can generally be of any convenient size and fabricated from any number of known materials. Preferably, the solid support used in the embodiments disclosed herein can be of any suitable type that provides a known binding capacity, resulting in a substantially unfluctuating amount of bound nucleic acids per fixed amount of solid support. Examples of such materials include: inorganics, natural polymers, and synthetic polymers. Specific examples of these materials include: cellulose, cellulose derivatives, acrylic resins, glass, silica gels, gelatin, polystyrene, polyvinyl pyrrolidone, co-polymers of vinyl and acrylamide, polystyrene cross-linked with divinylbenzene or the like, polyacrylamides, latex gels, silicon, plastics, nitrocellulose, polystyrene, dextran, rubber, natural sponges, silica gels, control pore glass, metals, cross-linked dextrans (e.g., Sephadex™) agarose gel (Sepharose™), and other solid supports known to those of skill in the art.

In some embodiments, the solid phase supports can include synthetic polymer supports, such as polystyrene, polypropylene, substituted polystyrene (e.g., carboxylated or aminated polystyrene), polyamides, polyacrylamides, polyvinylchloride, and the like, or any material useful in nucleic acid affinity chromatography. In some embodiments, the solid phase can be a flat surface, curved surface, a well, or part of a microfluidic device.

In some embodiments disclosed herein, the solid support can include beads. Beads may be any of a wide variety of shapes, such as spherical, generally spherical, egg shaped, disc shaped, cubical, amorphous and other three dimensional shapes. Beads may be manufactured using a wide variety of materials, including for example, resins, and polymers.

This allows the removal of the negatively charged compounds from the extract (chlorophyll lacking the phytol group and the fatty acids) by removing the immobilized base from the extract. In one embodiment, the strong base is a quaternary amine. The term “quaternary amine”, as used herein, refers to positively charged polyatomic ions comprising the structure NR+4 wherein R is an alkyl group or an aryl group. Quaternary amines are usually formed by reaction between a basic nitrogen of a compound and an appropriate quaternizing agent, such as, for example, an optionally substituted alkylhalide, arylhalide or arylalkylhalide, e.g. methyliodide or benzyliodide. Suitable quaternary amines for use in the present invention include, without limitation, salts of the tetramethylammonium ion, salts of the tetraethylammonium ion, salts of the tetrapropylammonium ion or salts of the tetrabutylammonium ion. In some embodiments, the immobilization is achieved by coupling the strong base to beads.

In accordance with any of the embodiments described herein, the strong base may be coupled to a support.

In step a(iv), the material obtained in step (iii) is treated under oxidative conditions. As used herein, the term “oxidative conditions” involve the contacting of the material with an oxidant. By the term oxidant is meant an agent (oxidant, oxidizer), or oxidizing agent (oxidizer) being a substance that has the ability to oxidize other substances, in other words to accept their electrons. Common oxidizing agents are oxygen, hydrogen peroxide, ozone and halogen-containing compounds such as sodium hypochlorite. An oxidizing agent is a chemical species that undergoes a chemical reaction in which it gains one or more electrons. In that sense, it is one component in an oxidation/reduction (redox) reaction. In the second sense, an oxidizing agent is a chemical species that transfers electronegative atoms, usually, oxygen to a substrate.

The second step (step (b)) of the method according to the invention is carried out in three different substeps, namely:
(i) disrupting the cell wall of the second algal biomass,
(ii) recovering the alkali-insoluble fraction from the fraction obtained in step (i) and
(iii) solubilizing the alkali-insoluble fraction in acidic media.

In a first step, the cells of the second algal biomass are treated so as to disrupt the cell wall, which leads to a lysed algal cell composition. As used herein, the terms “lyse” and “lysing” refer to a process of rupturing the cell wall and/or cell membrane of a cell Disruption of the cell wall can be carried out by different means, such as mechanically treatment, chemical treatment, enzymatic treatment, physical treatment, or combinations thereof. In various embodiments, the lysis occurs by the addition of an enzyme capable of disrupting the cell wall of the microbial cells. Disruption of the cell wall leads to a lysed cell composition. The processes of the present invention comprise lysing an algal cell composition or algal cell biomass to form a lysed algal cell composition.

In one embodiment, the disruption of the cell wall is carried out under mild alkaline conditions. This results in the formation of an insoluble fraction which contains the polysaccharides and that can be recovered by any means that allows the separation of a soluble from insoluble fraction, such as filtration or centrifugation. In step (iii) of the process, the alkali-insoluble fraction containing the polysaccharides can then be isolated by solubilization in acidic media so as to obtain a polysaccharide-rich fraction wherein the polysaccharides are found in soluble form.

Solubilization of the alkali-insoluble material requires decreasing of the pH, which can be done by using organic or inorganic acids like sulfuric acid, nitric acid, phosphoric acid, boric acid, hydrochloric acid, hydrobromic acid, perchloric acid, hypochlorous acid, chlorous acid, fluorosulfuric acid, hexafluorophosphoric acid, acetic acid, citric acid, formic acid, or combinations thereof. Due to easiness of handling, the acids and bases are preferably used in liquid form, in particular as concentrated solutions, wherein the concentration of acid or base in the solution is preferably in the range of 10 to 55 wt.-percent, in particular in the range of 20 to 50 wt.-percent.

The amount of acid used in step (b)(iii) is sufficient to maintain a pH of at least about 7.0, e.g. at least about 6.5, 6.0, 5.5, 4.0, 4.5, 3.0, 2.5, 2.0, 1.5, 1.0, 0.5, in the mixture for the duration of step (b)(iii). In certain embodiments, the reaction is allowed to proceed until substantially, e.g. essentially, all of the polysaccharides have been solubilized.

In some embodiments, the acid is added in an amount of about 2 percent to about 10 percent, about 2 percent to about 9 percent, about 2 percent to about 8 percent, about 2 percent to about 7 percent, about 2 percent to about 6 percent, about 3 percent to about 6 percent, about 4 percent to about 6 percent, about 5 percent to about 6 percent, about 2 percent to about 5 percent, about 2 percent to about 4 percent, about 2 percent to about 3 percent, about 3 percent to about 5 percent, about 3 percent to about 4 percent, or about 4 percent to about 5 percent by weight (or volume) of the solution to decrease the pH.

### A Foodstuff, Cosmeceutical, Cosmetic, Nutraceutical, Nutritional Supplement or Pharmaceutical Composition

The combination of the invention can be present in a foodstuff.

Foodstuff as used herein relates to a substance that can be used or prepared for use as food. The term also relates to any substance or product, whether processed, partially processed or unprocessed, intended to be, or reasonably expected to be ingested by humans. These compositions can also be named as terms “food product”, “food composition”, food ingredient or food additive. These compositions can also relate to a food that beneficially affects one or more functions of the body, so as to provide better health and wellness. Accordingly, such a food product may be intended for the prevention and/or treatment of a disease or a disease causing factor. Therefore, these compositions can also be named as functional food for particular nutritional purposes. The foodstuff can be a ready-to-eat-food. A ready-to-eat food is that which does not need to be diluted by means of an aqueous solution suitable for consumption for example. In principle, the ingredients present in a ready-to-eat food are balanced and there is no need to add additional ingredients to the food to make it ready to eat, such considered by a person skilled in the art. A concentrated food is that in which one or more ingredients are present at a higher concentration than in a ready-to-eat food, therefore for use it is necessary to dilute it by means of an aqueous solution suitable for consumption for example. Non-limiting, illustrative examples of foods provided by this invention include both dairy products and derivatives, for example, fermented milks, yoghurt, kephir, curd, cheeses, butters, ice creams, milk-based desserts, etc., and non-dairy products, such as baked products, cakes and pastries, cereals, chocolates, jams, juices, other fruit derivatives, oils and margarines, prepared dishes, etc.

In another particular embodiment, the product of the invention is a nutraceutical product comprising the combination of the invention and a nutraceutical acceptable carrier. Additionally the invention relates to the use of the combination of the invention as an ingredient in a nutraceutical product.

As used herein, the term “nutraceutical product” refers to a product suitable for use in human beings or animals, comprising one or more natural products with therapeutic action which provide a health benefit or have been associated with disease prevention or reduction, and it includes dietary supplements presented in a non-food matrix (e.g., capsules, powder, etc.) of a concentrated natural bioactive product usually present (or not) in the foods and which, when taken in a dose higher than that existing in those foods, exerts a favorable effect on health which is greater than effect which the normal food may have. Therefore, the term “nutraceutical product” includes isolated or purified food products as well as additives or food supplements which are generally presented in dosage forms normally used orally, for example, capsules, tablets, sachets, drinkable phials, etc.; such products provide a physiological benefit or protection against diseases, generally against chronic diseases. If desired, the nutraceutical product provided by the invention can contain, in addition to the combination of the invention, one or more nutraceuticals (products or substances associated with disease prevention or reduction), for example, flavonoids, omega-3 fatty acids, etc., and/or one or more prebiotics (non-digestible food ingredients which stimulate probiotic activity and/or growth), for example, oligofructose, pectin, inulin, galacto-oligosaccharides, lactulose, human milk oligosaccharides, dietary fiber, etc.

In another particular embodiment, the product of the invention is a cosmeceutical product comprising the combination of the invention and a cosmeceutical acceptable vehicle or carrier. Additionally, the invention relates to the use of the combination of the invention as an ingredient in a cosmeceutical product.

As used herein, the term “cosmeceutical product” refers to a product suitable for use in the body or animal body comprising one or more cosmeceutical products (functional cosmetics, dermaceuticals or active cosmetics), i.e., topical hybrid products with cosmetic-pharmaceutical characteristics containing active ingredients having effect on user's skin, hair and/or nails, at higher and more effective concentrations, therefore they are located in an intermediate level between cosmetic and drug. Illustrative examples of cosmeceutical products include essential oils, ceramides, enzymes, minerals, peptides, vitamins, etc.

The invention relates to a cosmetic composition comprising the combination of the invention and a cosmetic acceptable carrier or vehicle. The invention also relates to the use of the combination of the invention for use as an ingredient in a cosmetic formulation.

“Cosmetic composition”, as used herein refers to a composition suitable for use in personal hygiene of human beings or animals, or in order to enhance the natural beauty or change the body appearance without affecting the structure or functions of the human or animal body, comprising one or more products providing such effects. If desired, the cosmetic composition provided by the invention can contain, in addition to the combination of the invention, one or more cosmetics or cosmetic products, i.e., substances or mixtures intended to be placed in contact with the external parts of the human or animal body (e.g., epidermis, hair system, nails, lips, etc.) or with the teeth and the buccal mucosa, for the exclusive or main purpose of cleaning them, perfuming them, changing their appearance, protecting them, keeping them in good condition or correcting body odors. Illustrative examples of cosmetically acceptable vehicles include the products contained in the INCI (International Nomenclature of Cosmetic Ingredients) list. The combination of the present invention may be added to a wide variety of products for cosmetic application, including makeup, creams for cleansing, protecting, treating, or caring for the skin, in particular, the face, hands, and feet (e.g., day and night creams, makeup removal creams, foundation creams and sunscreens), liquid foundations, makeup removal lotions, protective or skin-care body lotions, sunscreen lotions, skin care lotions, gels, or foams, such as cleansing, sunscreen, and artificial tanning lotions, bath preparations, deodorant compositions, after-shave gels or lotions, depilatory creams, and compositions used for insect stings and against pain. The combination of the invention may take any of a wide variety of forms, and include, for example dressings, lotions, solutions, sprays, creams, gels, ointments, or the like.

In addition, the invention relates to a pharmaceutical product comprising the combination of the invention and a vehicle or carrier suitable for oral, topical or parenteral administration. The invention also relates to the use of the combination of the invention as an ingredient in a pharmaceutical composition.

“Pharmaceutical composition”, as used herein, relates to compositions and molecular entities that are physiologically tolerable. Preferably, the term “pharmaceutically acceptable” means it is approved by a regulatory agency of a state or federal government or is included in the U.S. Pharmacopeia or other generally recognized pharmacopeia for use in animals, and more particularly in humans.

The pharmaceutical acceptable carriers or vehicles are well-known to those skilled in the art and are readily available to the public.

Based on the particular mode of administration, the pharmaceutical product may be formulated into solid, liquid, injectable or topical dosage forms.

Solid dosage forms for oral administration may include conventional capsules, sustained release capsules, conventional tablets, sustained-release tablets, chewable tablets, sublingual tablets, effervescent tablets, pills, suspensions, powders, granules and gels. At these solid dosage forms, the active compounds can be mixed with at least one inert excipient such as sucrose, lactose or starch. Such dosage forms can also comprise, as in normal practice, additional substances other than inert diluents, e.g. lubricating agents such as magnesium stearate. In the case of capsules, tablets, effervescent tablets and pills, the dosage forms may also comprise buffering agents. Tablets and pills can be prepared with enteric coatings.

Liquid dosage forms for oral administration may include emulsions, solutions, suspensions, syrups and elixirs pharmaceutically acceptable containing inert diluents commonly used in the technique, such as water. Those compositions may also comprise adjuvants such as wetting agents, emulsifying and suspending agents, and sweetening agents, flavoring and perfuming agents.

Injectable preparations, for example, aqueous or oleaginous suspensions, sterile injectable may be formulated according with the technique known using suitable dispersing agents, wetting agents and/or suspending agents. Among the acceptable vehicles and solvents that can be used are water, Ringer's solution and isotonic sodium chloride solution. Sterile oils are also conventionally used as solvents or suspending media.

For topical administration the pharmaceutical composition of the invention can be formulated as creams, gels, hydrogel, lotions, liquids, pomades, spray solutions, dispersions, solid bars, emulsions, microemulsions and similars which may be formulated according to conventional methods that use suitable excipients, such as, for example, emulsifiers, surfactants, thickening agents, coloring agents and combinations of two or more thereof.

All the particular embodiments of the method of the present invention are also applicable to this aspect of the invention.

### Non-Therapeutic Uses

“Antioxidant composition”, as used herein relates to a composition which reduces the amount of oxidation over a given period when compared to the oxidation that would occur in the absence of that composition or it is a meant a material which increase the time required for a given amount of oxidation to occur when compared to the oxidation that would occur in the absence of that composition.

The antioxidant activity can be determined by means of any known assay such as DPPH (2,2-diphenyl-1-picrylhydrazyl) and ABTS tests. The antioxidant capacity can also be determined by measuring the ability of antioxidant compounds to react with a given free radical, or determining that such compounds would have potential to reduce the complex formed between Fe (III) ions and the reagent TPTZ (2, 4,6-tripyridyl-s-triazine). Among those tests that rely on measuring the ability of antioxidants to react with a free radical, ORAC test (Oxygen Radical Absorbance Capacity Oxygen Radical Absorbance Capacity), TEAC assay (Trolox Equivalent Antioxidant Capacity or as Trolox equivalent antioxidant capacity). In addition, the Radical Scavenging Index (RSI) a measure of radical scavenging capacity can be used for determining the antioxidant capacity. In a preferred embodiment, the antioxidant capacity is determined by DPPH method (RSA, Radical Scavenging Activity).

All the particular embodiments of the method of the present invention are also applicable to this aspect of the invention.

### Medical Uses

All the particular embodiments of the method of the present invention are also applicable to this aspect of the invention.
* * *

This invention is further illustrated by the following examples, which are not to be construed in any way as imposing limitations upon the scope thereof.

### EXAMPLES

### Materials

Chlorella vulgaris Extract Obtaining, Purification and Encapsulation

Chlorella vulgaris dry biomass is extracted with alcohol and filtered. The resulting organic extract was purified by means of a strong basic anionic resin in order to selectively remove chlorophylls and esterified fatty acids. The resin is separated from the extract by filtration and the purified extract was encapsulated in liposomes by high pressure homogenization after evaporating the organic solvent.

### Apocarotenoid Formation

The apocarotenoid formation was adapted from Henry et al. (J. Agric. Food Chem. 2000, 48, 5008-5013). A gentle synthetic air flow (20% oxygen) is allowed to pass through this encapsulated Chlorella vulgaris fraction until the desired amount of oxidation products is reached, determined by HPLC. Then, the solution is acidified before combining with the polysaccharide fraction.

### Polysaccharide Fraction Extraction

Chlorella vulgaris cell wall disruption was performed under mild alkaline conditions. Both supernatant and alkali-insoluble fraction were separated by centrifugation and the pellet was washed with water several times and dried to obtain the polysaccharide fraction.

This alkali-insoluble polysaccharide fraction is solubilized in acidic media in order to prepare the combination.

### Combination Preparation

The encapsulated apocarotenoid-containing extract is mixed with a desired amount of polysaccharide fraction solution to obtain the desired combination.

### Apocarotenoid Content Determination

A sample of the encapsulated oxidized Chlorella fraction is extracted with ethyl acetate. The organic solvent is evaporated and the residue redissolved in acetone. The lutein and chlorophyll content is quantified by HPLC at 450 nm using their corresponding standard reference compounds. The apocarotenoid total content is calculated using the following formula:

$$( {Q}_{lutein encap} - {Q}_{lutein oxid} ) / {Q}_{lutein encap} \times 100 = \% total amount apocarotenoids$$

Where Q_{lutein encap} is the content of lutein by HPLC at 450 nm before oxidation and Q_{lutein oxid} is the content of lutein after oxidation.

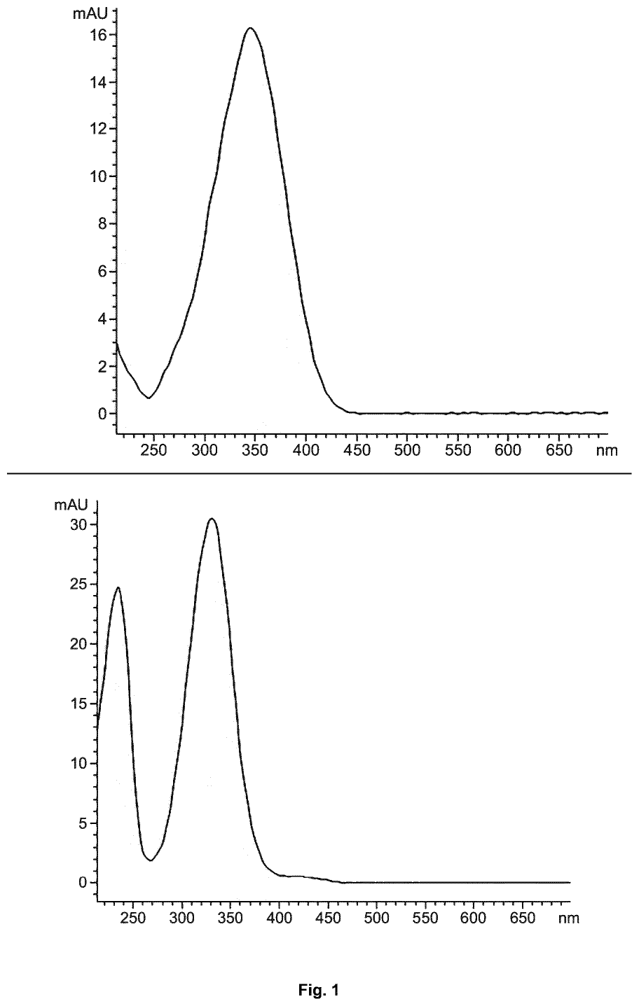

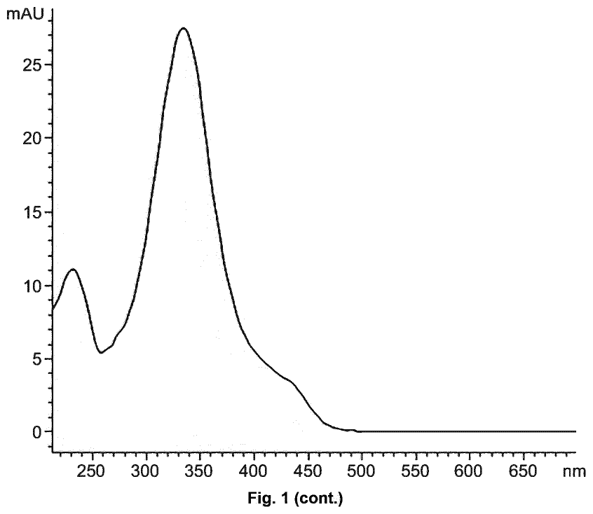

Some examples of UV spectra of the detected apocarotenoids at 300-350 nm are shown in FIG. 1. These UV spectra would go in agreement with the described spectra by Kopec et al. (J.I Chromatography B, 1102-1103, 45-51).

### Polysaccharide Determination

Several methods are used in order to determine the polysaccharide fraction. Dubois et al (Anal. Chem. 1956, 28, 3, 350-356) was used as a colorimetric method for sugar determination. For monosaccharide composition, a HPLC method was used as described in Blanco-Gomis et al. (Anal Chim Acta. 2001, 436, 173-180). Glucosamine analysis was performed as described in Yan et al. (Carbohydrate Polymers, 2012, 87, 2, 1774-1778).

### Assays for the Determination of RAR Agonistic Activity

An agonist assay for RAR was performed by SelectScreen® Cell-Based Nuclear Receptor Profiling Service of Life Technologies. In this assay, an engineered cell line (UAS-bla HEK 293T) expressing RARalpha, RARbeta or RAR gamma is used to test the action of lutein. GeneBLAzer® technology, based on mammalian-optimized Beta-lactamase reporter gene (bla) combined with a FRET-enabled substrate, provides the assay with a sensitive detection method.

### Example 1—Specific Agonistic Activity of Lutein for Alpha, Beta and Gamma RAR

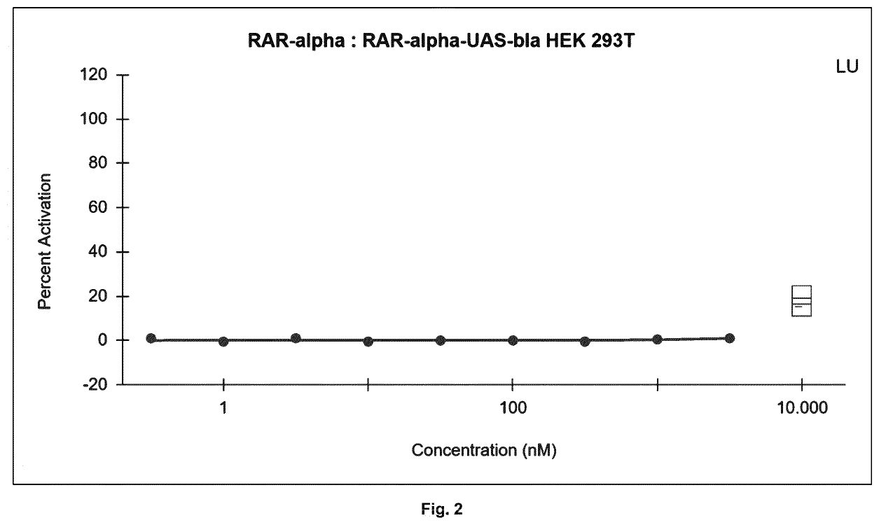

RAR-alpha-UAS-bla HEK 293T cells are thawed and resuspended in Assay Media (DMEM phenol red free, 2% CD-treated FBS, 0.1 mM NEAA, 1 mM Sodium Pyruvate, 100 U/mL/100 μg/mL Pen/Strep) to a concentration of 312,500 cells/mL. 4 μL of a 10× serial dilution of ATRA (control agonist starting concentration, 10 nM) or test compound (Lutein) are added to appropriate wells of a 384-well Poly-D-Lysine assay plate. 32 μL of cell suspension (10,000 cells) is added to each well. 4 μL of Assay Media is added to all wells to bring the final assay volume to 40 μL. The plate is incubated for 16-24 hours at 37° C./5% CO2 in a humidified incubator. 8 μL of 1 μM Substrate Loading Solution is added to each well and the plate is incubated for 2 hours at room temperature. The plate is read on a fluorescence plate reader. The results are shown in FIG. 2. No agonist effect of Lutein could be measured at any concentration below 3.2 μM.

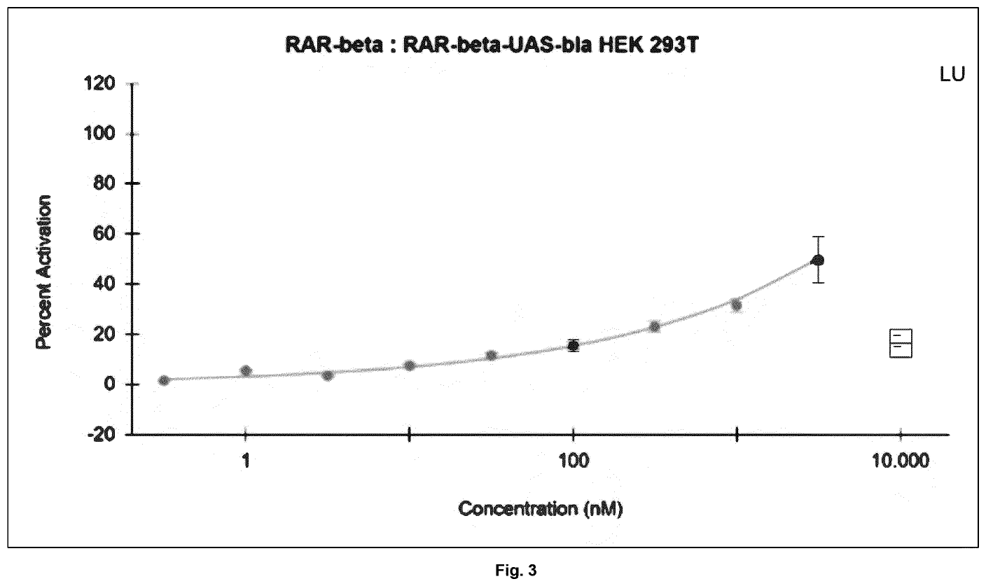

RAR-beta-UAS-bla HEK 293T cells are thawed and resuspended in Assay Media (DMEM phenol red free, 0.1% BSA, 0.1 mM NEAA, 1 mM Sodium Pyruvate, 100 U/mL/100 μg/mL Pen/Strep) to a concentration of 312,500 cells/mL. 4 μL of a 10× serial dilution of ATRA (control agonist starting concentration, 10 nM) or test compound (Lutein)s are added to appropriate wells of a 384-well TC-Treated assay plate. 32 μL of cell suspension (10,000 cells) is added to each well. 4 μL of Assay Media is added to all wells to bring the final assay volume to 40 μL. The plate is incubated for 16-24 hours at 37° C./5% CO2 in a humidified incubator. 8 μL of 1 μM Substrate Loading Solution is added to each well and the plate is incubated for 2 hours at room temperature. The plate is read on a fluorescence plate reader. The results are shown in FIG. 3. A specific RAR-beta partial agonist effect of Lutein could be measured for concentrations above 100 nm, reaching approximately a 40% activation at 3.16 μM.

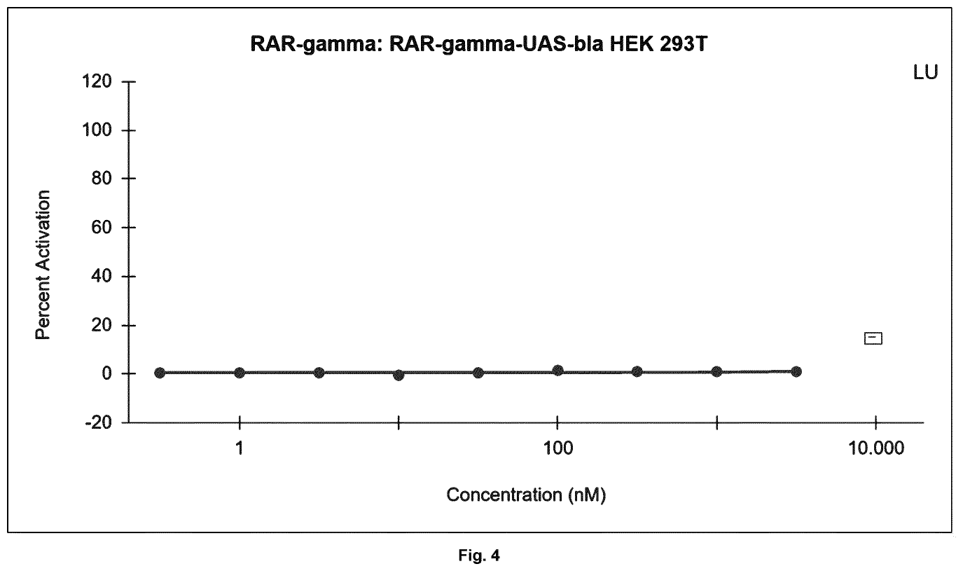

RAR-gamma-UAS-bla HEK 293T cells are thawed and resuspended in Assay Media (DMEM phenol red free, 2% CD-treated FBS, 0.1 mM NEAA, 1 mM Sodium Pyruvate, 100 U/mL/100 μg/mL Pen/Strep) to a concentration of 156,250 cells/mL. 4 μL of a 10× serial dilution of ATRA (control agonist starting concentration, 10 nM) or test compound (Lutein)s are added to appropriate wells of a 384-well TC-Treated assay plate. 32 μL of cell suspension (5,000 cells) is added to each well. 4 μL of Assay Media is added to all wells to bring the final assay volume to 40 μL. The plate is incubated for 16-24 hours at 37° C./5% CO2 in a humidified incubator. 8 μL of 1 μM Substrate Loading Solution is added to each well and the plate is incubated for 2 hours at room temperature. The plate is read on a fluorescence plate reader. The results are shown in FIG. 4. No agonist effect of Lutein could be measured at any concentration below 3.2 μM.

### Example 2—Efficacy of the Cosmetic Product

Aim of the study is to evaluate the efficacy of an active cosmetic product for face area in reducing the skin wrinkledness and improving skin elasticity, firmness and uneven complexion in comparison to a reference product (composed by retinol 0.3%). In order to reach this goal a randomized controlled clinical instrumental study is carried out on 30 healthy male and female subjects aged over 40 years old, showing clinical sign of skin aging such as fine lines/wrinkles in the crow's feet and uneven skin tone due to dark spots. According to a previously defined randomization list the included subjects apply the test product on one face side and the reference product on the contralateral side. Evaluations are performed at baseline (T0) and after 7 (T7), 14 (T14), 28 (T28) and 56 (T56) days of products use. Instrumental measurements are carried out by means of non-invasive bioengineering techniques and they are integrated with the clinical analysis carried out by the Dermatologist and the self-assessment filled in by the volunteers.

Test product: Chlorella extract containing 0.00005% w/w lutein and 0.1% polysaccharides w/w encapsulated in liposomes. Formulated at 1% v/v in Aqua, Coco-Caprylate/Caprate, Glycerin, Phenoxyethanol, Ethylhexylglycerin, Sodium Acrylate/Sodium Acryloyldimethyl Taurate Copolymer, Hydrogenated Polydecene, Trideceth-6, Sorbitan Laurate, Chlorella Vulgaris Extract, Lecithin, Lactic Acid, Disodium EDTA.

Reference product: Retinol. Formulated at 0.3% in Aqua, Coco-Caprylate/Caprate, Glycerin, Phenoxyethanol, Ethylhexylglycerin, Sodium Acrylate/Sodium Acryloyldimethyl Taurate Copolymer, Hydrogenated Polydecene, Trideceth-6, Sorbitan Laurate, Caprylic/Capric Triglyceride, Retinol, Disodium EDTA.

Way of use: both products are applied twice a day (morning and afternoon) on the assigned half face massaging for one to two minutes.

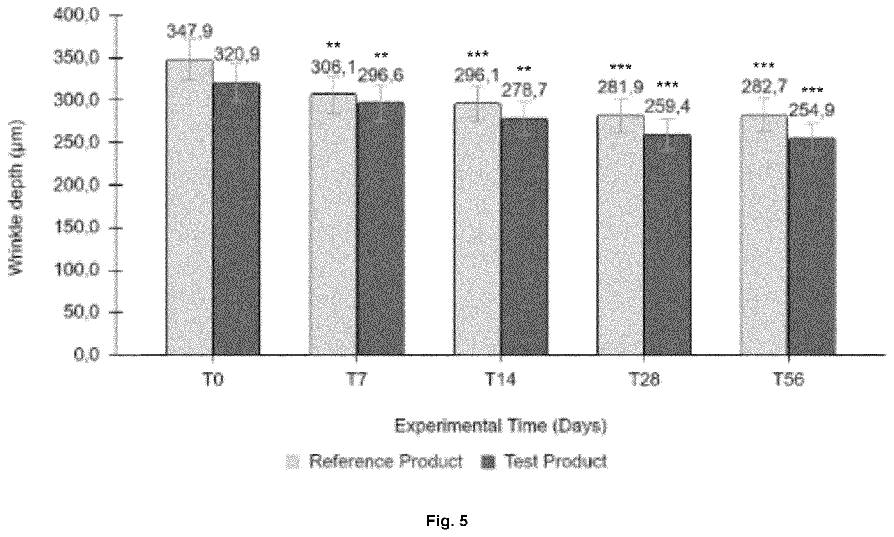

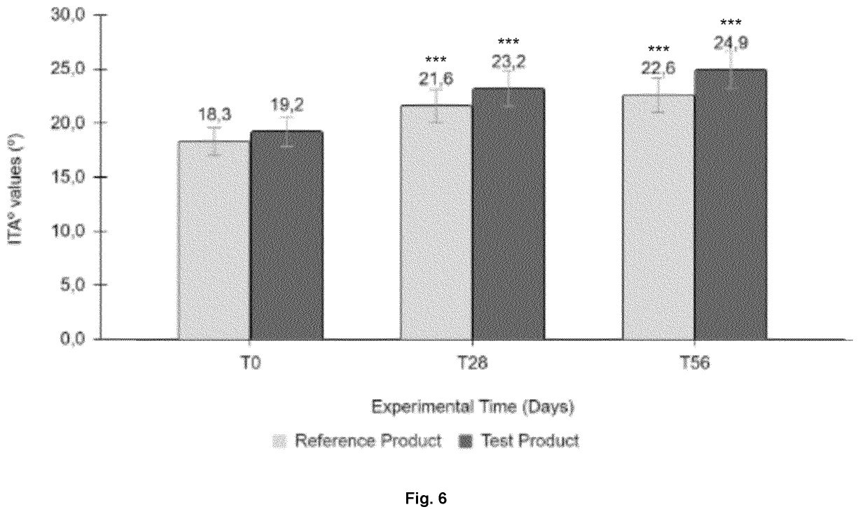

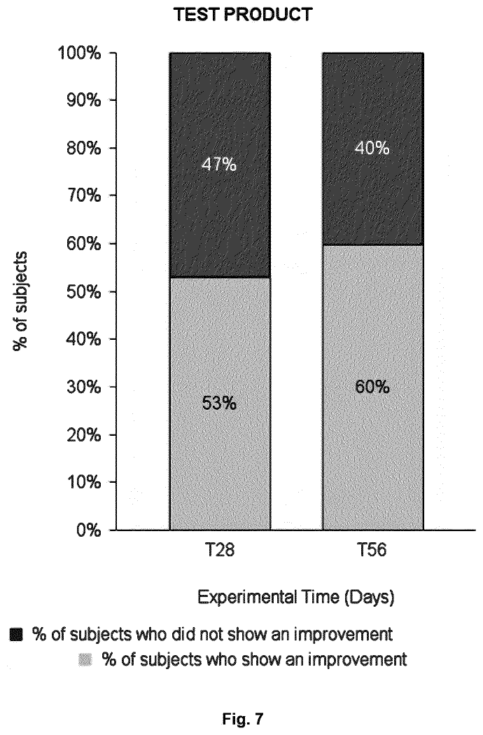

The results of the skin profilometry (wrinkle depth), the evaluation of the intensity of dark spot colour, calculation of ITA° and the Clinical evaluation of the skin complexion evenness are shown in FIGS. 5, 6 and 7.

### CONCLUSIONS

The clinical analysis carried out by the dermatologist highlights:
an improvement of dark/age spots colour in 63% of the enrolled subjects at T28 and in 67% of them at T56;
an improvement of skin complexion evenness in 53% of the enrolled subjects at T28 and in 60% of them at T56.

No significant difference is observed between the two tested products.

During the study period the products were well tolerated by all the subjects participating in the study and positively judged by most of them for all the investigated aspects (erythema, oedema, desquamation, itching or burning feeling not observed among others.)

1. A combination comprising lutein and a polysaccharide, wherein the combination is characterized in that it has
(i) a content of apocarotenoids which is of at least 0.00005% (w/w) and/or
(ii) a content of chlorophyll which is below 0.05% (w/w).

2. The combination according to claim 1, wherein the polysaccharide is selected from the group consisting of starch, cellulose, a galacturonic acid-rich polysaccharide and a glucosamine-rich polysaccharide.

3. The combination according to claim 2, wherein the galacturonic acid-rich polysaccharide is pectin or wherein the glucosamine-rich polysaccharide is chitosan, chitin or a combination thereof.

4. The combination according to claim 1 wherein the content of lutein in the combination is of at least 0.00005% (w/w).

5. (canceled)

6. (canceled)

7. (canceled)

8. The combination according to claim 1, wherein the content of polysaccharides in the combination is at least 90% (w/w).

9. (canceled)

10. (canceled)

11. The combination according to claim 1, wherein the content of apocarotenoids in the combination is between 0.00005%-5% (w/w).

12. (canceled)

13. (canceled)

14. The combination according to claim 1, wherein
(i) the combination is a composition containing liposomes and polysaccharides wherein the liposomes encapsulate the lutein and the apocarotenoids but do not encapsulate the polysaccharides,
(ii) the combination is a composition comprising liposomes, wherein liposomes encapsulate the lutein, the apocarotenoids and the polysaccharides or
(iii) the combination is a composition comprising a first population of liposomes in which the lutein and apocarotenoids are encapsulated and a second population of liposomes in which the polysaccharides are encapsulated.

15. (canceled)

16. A process for preparing a combination according to claim 1 comprising the steps of:
a. Preparing a first extract containing lutein and apocarotenoids from a first algal biomass preparation by a process comprising the steps of:
(i) Extracting the first algal biomass with an organic solvent,
(ii) Applying saponification conditions to the extract obtained in step (i),
(iii) Removing negatively charged compounds resulting from the saponification carried out in step (ii) and
(iv) Treating the fraction obtained in step (iii) under oxidative conditions in order to convert part of the carotenoids found in the fraction to apocarotenoids,
b. Preparing a second extract containing polysaccharides from a second algal biomass preparation by a process comprising the steps of:
(i) disrupting the cell wall of the second algal biomass,
(ii) recovering the alkali-insoluble fraction from the fraction obtained in step (i) and
(iii) solubilizing the alkali-insoluble fraction in acidic media and
c. Combining the fractions obtained in steps a. and b.

17. The process according to claim 16 wherein the algal biomass is from a species of the genus Chlorella.

18. The process according to claim 17 wherein the species of the genus Chlorella is Chlorella vulgaris.

19. (canceled)

20. The process according to claim 16 wherein the saponification conditions in step a(ii) are created by adding an immobilized strong base to the extract and wherein the removal of negatively charged compounds in step a(iii) is carried out by removing the immobilized base from the extract.

21. (canceled)

22. The process according to claim 16 wherein the oxidative conditions used in step a(iv) include exposing the material obtained in step a(iiI) to oxygen or ozone.

23. (canceled)

24. A combination obtainable by the method according to claim 16.

25. A foodstuff, cosmeceutical, cosmetic, nutraceutical, nutritional supplement or pharmaceutical composition comprising the combination according to claim 1 and a vehicle adequate for the formulation of the combination into said foodstuff, cosmeceutical, nutraceutical composition, cosmetic composition nutritional supplement or pharmaceutical composition.

26. A cosmetic method for preventing and/or reducing the cosmetic adverse effects of aging comprising administering the foodstuff, cosmeceutical, nutritional supplement, nutraceutical or cosmetic composition according to claim 25 to a subject in need thereof.

27. (canceled)

28. A cosmetic method for improving skin elasticity and firmness comprising administering the foodstuff, cosmeceutical, nutraceutical, nutritional supplement or cosmetic composition according to claim 25 to a subject in need thereof.

29. (canceled)

30. (canceled)

31. A pharmaceutical composition comprising a combination according to claim 1 and a pharmaceutically acceptable adjuvant.

32. (canceled)
